(19) United States
(12) Patent Application Publication
(10) Pub. No.: US 2026/0315294 A1
(43) Pub. Date: Oct. 15, 2026

(54) Menstrual hygiene system with integrated disposal bags and optional scented bags.

(71) Applicant: VANESSA WALLEN, TORRINGTON, CT (US)

(72) Inventor: VANESSA WALLEN, TORRINGTON, CT (US)

(21) Appl. No.: 19/417,661

(22) Filed: Dec. 12, 2025

Related U.S. Application Data
(60) Provisional application No. 63/733,824, filed on Dec. 13, 2024.

(51) Int. Cl. A61F 13/551

(52) U.S. Cl. CPC A61F 13/5515; A61F 13/5514; A61F 13/5518; A61F 2013/55155

(57) ABSTRACT

A menstrual hygiene system is provided in which each individual tampon wrapper and each individual pad wrapper includes an attached, removable disposal bag configured to receive a used tampon or pad. The disposal bag is joined to an exterior surface of the wrapper along a tear line so that the user can separate the bag, open it, and enclose the used product for discrete disposal. The attached bags are typically unscented for general use, while scented disposal bags may be supplied separately as optional accessories.

### BACKGROUND

Field of the invention. The present invention relates to menstrual hygiene products and, more particularly, to tampon and pad packaging having an integrated disposal bag for discreet disposal of used products.

Users of tampons and disposable pads often lack a convenient, discreet receptacle for sealing used products, especially outside the home. Some products provide stand-alone disposal bags sold separately, but they are not supplied on a per-unit basis with each wrapped tampon or pad as an integrated system. There remains a need for a menstrual product system in which each individually wrapped product includes its own attached disposal bag, while scented bags may be provided separately as an optional accessory

### SUMMARY OF THE INVENTION

The invention provides a menstrual hygiene system in which each individual tampon wrapper and each individual pad wrapper carries an attached, removable disposal bag for receiving the used product. The attached bags are typically unscented, while scented disposal bags are offered for separate sale as accessories.

In one aspect, a tampon product includes a tampon enclosed within an individual wrapper, and a folded disposal bag attached to an exterior region of the wrapper along a tear line. In another aspect, a pad product includes a disposable pad enclosed within an individual wrapper with a similar attached disposal bag. The disposal bag can be removed along the tear line, opened, and used to enclose the used tampon or pad for discreet disposal

### BRIEF DESCRIPTION OF THE DRAWINGS

FIG. 1 is a front view of an individual tampon wrapper having an attached, folded disposal bag

FIG. 2A is a view of a disposal bag in an open, unused state

FIG. 2B is a view of a disposal bag in a closed state after use

FIG. 3 is a front view of an individual pad wrapper having an attached, folded disposal bag

### DETAILED DESCRIPTION OF ILLUSTRATIVE EMBODIMENTS

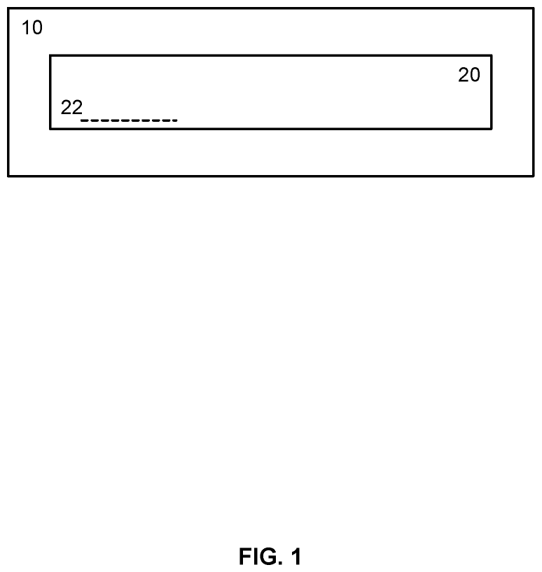

Referring now to FIG. 1, an individual tampon wrapper is generally designated by reference numeral 10. The wrapper 10 is a sealed package configured to enclose a tampon and its applicator (not shown in detail). Attached to an exterior surface of the wrapper 10 is a folded disposal bag 20. The disposal bag 20 is joined to the wrapper 10 along a tear line 22, which may be formed by perforations, a weakened region, or another frangible connection.

The disposal bag 20 is shown in FIG. 1 in a folded state suitable for compact packaging. When the user wishes to dispose of a used tampon, the user separates the bag 20 from the wrapper 10 along tear line 22 and unfolds the bag into an open configuration.

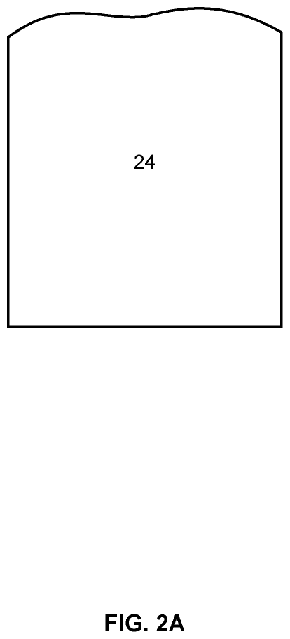

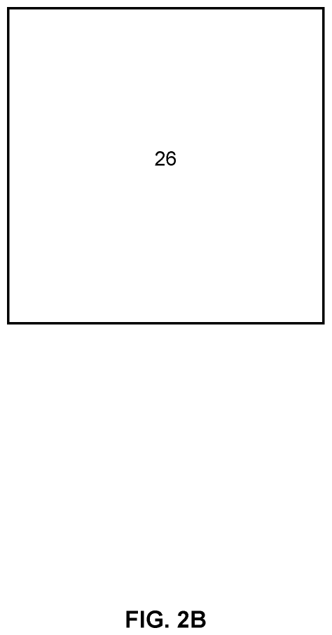

Turning to FIG. 2A, a disposal bag in an open state is generally designated by 24. The bag 24 has an open mouth at its upper end to receive a used tampon or pad. FIG. 2B shows the disposal bag in a closed state 26, after the used product has been placed inside. The bag 26 may be closed by tying, folding, an adhesive flap, or another closure mechanism to provide discreet containment of the used product.

In some embodiments, the bags 24, 26 are formed from an opaque, optionally biodegradable or compostable film to conceal the contents and to reduce environmental impact. The bags attached to individual wrappers are preferably unscented, while separately sold bags may include a fragrance.

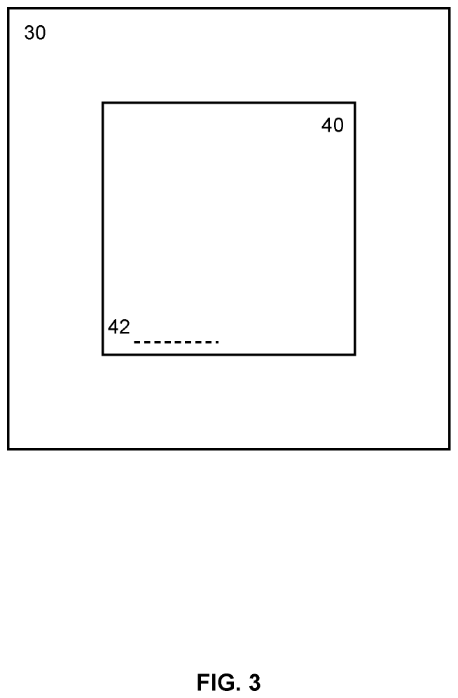

Referring to FIG. 3, an individual pad wrapper is generally designated by 30. The wrapper 30 encloses a disposable menstrual pad (not shown in detail). A folded disposal bag 40 is attached to an exterior surface of the wrapper 30 along a tear line 42, similar to bag 20 and tear line 22 discussed above. The user removes the bag 40 along tear line 42 and uses it to enclose the used pad in the same manner as described with respect to FIGS. 2A and 2B.

In use, each individually wrapped tampon or pad provides an immediately available, dedicated disposal bag, eliminating the need for users to carry separate bags or rely on restroom facilities. Scented disposal bags may be sold as a separate product line for users who desire additional odor control, while the wrapper-attached bags remain unscented

1. A menstrual hygiene system comprising:
(a) a plurality of tampons, each enclosed within an individual tampon wrapper;
(b) a plurality of disposable pads, each enclosed within an individual pad wrapper; and
(c) for each of the individual tampon wrappers and individual pad wrappers, a disposal bag attached to an exterior surface of the corresponding wrapper along a tear line, the disposal bag being removable from the wrapper and configured to receive and enclose a used tampon or pad;
wherein the disposal bags attached to the wrappers are unscented, and scented disposal bags are offered for separate sale as accessories.
